(19) United States
(12) Patent Application Publication
(10) Pub. No.: US 2024/0201150 A1
(43) Pub. Date: Jun. 20, 2024

(54) SYSTEM AND METHOD FOR DETERMINING GAS-WATER RATIO IN A RESERVOIR FLUID

(71) Applicant: SAUDI ARABIAN OIL COMPANY, Dhahran (SA)

(72) Inventors: Ahmed M. SAHL, Dammam (SA); Abdullah F. ALMOUSA, Al Jish (SA); Mohammed H. BASHRAWI, Al Qatif (SA); Daryl S. SEQUEIRA, Dhahran (SA)

(73) Assignee: SAUDI ARABIAN OIL COMPANY, Dhahran (SA)

(21) Appl. No.: 18/066,479

(22) Filed: Dec. 15, 2022

(51) Int. Cl. G01N 30/86; G01N 33/28

(52) U.S. Cl. CPC G01N 30/8651; G01N 30/8675; G01N 33/2841; G01N 33/2847; G01N 2030/025

(57) ABSTRACT

A gas-water flash separator system includes an inlet water line fluidly communicable with a sample cylinder containing a pressurized reservoir fluid, and a glass trap sealable with a manifold and in fluid communication with the inlet water line to receive the pressurized reservoir fluid, the pressurized reservoir fluid being separable within the glass trap at atmospheric conditions into a flashed gas and a reservoir water. A gasometer is in fluid communication with the glass trap via a gasometer flow line and operable to receive the flashed gas and measure a volume, a temperature, and a pressure of the flashed gas. A weight and a density of the reservoir water is measured and compared against the volume of the flashed gas to thereby determine a gas-water ratio of the pressurized reservoir fluid.

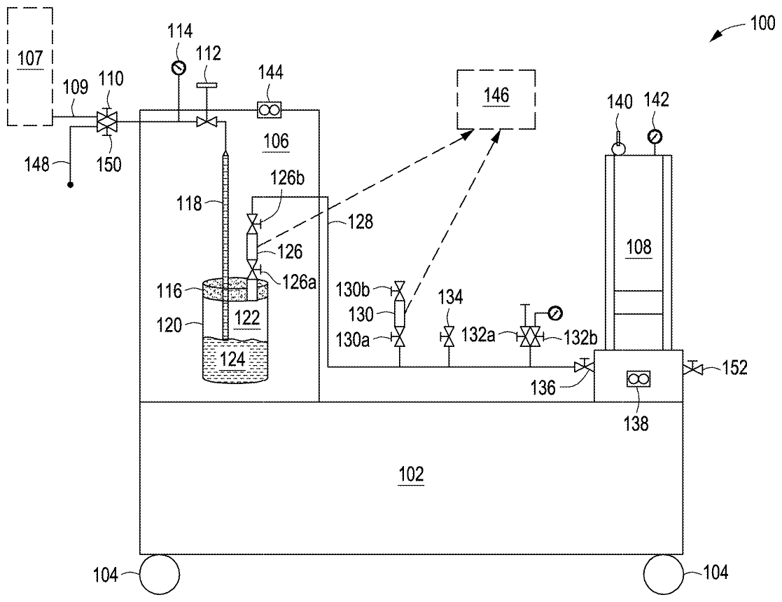

### FIELD OF THE DISCLOSURE

The present disclosure relates generally to analyzing reservoir fluids and, more particularly, to an apparatus, system, and method for determining gas-water ratio in a reservoir fluid.

### BACKGROUND OF THE DISCLOSURE

There are instances where it is desirable to determine the composition of a reservoir fluid, such as subterranean reservoir water, for reservoir management purposes, such as to determine gas-water ratios. The gas-water ratio is the ratio of gas produced from a barrel of produced water when cooled and depressurized to standard conditions; it is a measurement used to describe the volume of gas that is flashed from a barrel of produced water.

The gas-water ratio of a reservoir is a sensitive indicator of the behavior of an oil and gas reservoir connected to a water source, such as an aquifer or injected water source, and informs various wellbore exploration and development operations. Such exploration and development operations include drilling fluid composition operations; hydraulic fracturing operations; secondary oil recovery operations; aquifer pressure sustainment operations; water injection design due to unavailability of sea water; acid treatment decisions; and production design of tubings, flow lines, and surface facilities, among other such operations. It is accordingly important that the volume of gas measured is accurate, regardless of the particular concentration (e.g., high or low) within a water sample.

It can further be a challenge to obtain unpolluted, representative subterranean reservoir water samples. During drilling and completion operations, the zone closest to the wellbore is invaded by drilling mud and brines. Therefore, the first produced water will be a mixture of mud/brine and formation reservoir water. It is challenging to determine when representative reservoir water is produced. Moreover, water samples collected should be analyzed as soon as possible because the properties of samples change with time. Changes occur in temperature and pressure, gases evolve, pH changes, and precipitations occur. These changes have an effect on important ions, as well as certain physical properties.

Current gas-water ratio testing devices (e.g., flash separators) can lack significant sensitivity to measure low concentrations (volumes) of gasses present in reservoir water, such as hydrogen sulfide and/or carbon dioxide. Additionally, current gas-water ratio testing devices are located remotely from the wellsite itself. Indeed, depending on the location of the wellsite, such testing devices may be hundreds or thousands of miles from the wellsite, introducing considerable lag-time between sample collection and sample analysis, which introduce the aforementioned sample changes, can result in contamination, may discourage frequent testing or eliminate retesting, and introduce expense related to travel and time costs. Furthermore, current gas-water testing devices are not readily mobile, even in a remote setting.

Accordingly, there is a need for gas-water ratio testing at or near a wellsite and in the laboratory that is sensitive to detection of low gaseous concentrations in formation water samples and is easily connectable to gas chromatograph instrumentation.

### SUMMARY OF THE DISCLOSURE

Various details of the present disclosure are hereinafter summarized to provide a basic understanding. This summary is not an exhaustive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

According to an embodiment consistent with the present disclosure, a system is provided including an inlet water line fluidly communicable with a sample cylinder containing a pressurized reservoir fluid; a glass trap sealable with a manifold and in fluid communication with the inlet water line to receive the pressurized reservoir fluid, the pressurized reservoir fluid being separable within the glass trap at atmospheric conditions into a flashed gas and a reservoir water; and a gasometer in fluid communication with the glass trap via a gasometer flow line and operable to receive the flashed gas and measure a volume, a temperature, and a pressure of the flashed gas. A weight and density of the reservoir water is measured and compared against the volume of the flashed gas to thereby determine a gas-water ratio of the pressurized reservoir fluid.

According to an embodiment consistent with the present disclosure, a system is provided including a base including a plurality of wheels. An enclosed flash separator assembly is positioned on the base and includes an inlet water line fluidly communicable with a sample cylinder containing a pressurized reservoir fluid; a glass trap scalable with a manifold and in fluid communication with the inlet water line to receive the pressurized reservoir fluid, the pressurized reservoir fluid being separable within the glass trap at atmospheric conditions into a flashed gas and a reservoir water; and a gasometer positioned on the base and in fluid communication with the glass trap via a gasometer flow line, the gasometer being operable to receive the flashed gas and measure a volume, a temperature, and a pressure of the flashed gas. A weight and density of the reservoir water is measured and compared against the volume of the flashed gas to thereby determine a gas-water ratio of the pressurized reservoir fluid.

According to an embodiment consistent with the present disclosure, a method is provided including fluidly coupling a sample cylinder containing a pressurized reservoir fluid to an inlet water line; receiving the pressurized reservoir fluid in the inlet water line, and conveying the pressurized reservoir fluid to a glass trap sealed with a manifold; separating the pressurized reservoir fluid in the glass trap at atmospheric conditions into a flashed gas and a reservoir water; receiving the flashed gas in a gasometer flow line in fluid communication with the glass trap via a gasometer flow line; measuring a volume, a temperature, and a pressure of the flashed gas with the gasometer; measuring a weight and a density of the reservoir water; and comparing the weight and the density against the volume of the flashed gas and thereby determining a gas-water ratio of the pressurized reservoir fluid.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

The FIGURE shows an illustrative schematic view of an example system of a mobile gas-water flash separator apparatus according to one or more aspects of the present disclosure.

### DETAILED DESCRIPTION

Embodiments of the present disclosure will now be described in detail with reference to the accompanying FIGURES. Like elements in the various FIGURES may be denoted by like reference numerals for consistency. Further, in the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying FIGURES may vary without departing from the scope of the present disclosure.

The present disclosure relates generally to analyzing reservoir fluids and, more particularly, to an apparatus, system, and method for determining gas-water ratio in a reservoir fluid. As described herein, a mobile gas-water flash separator apparatus is disclosed and is used to optimize the separation of gas and water, is capable of measuring small concentrations (i.e., volumes) of gas, and captures gas liberated from water for effective chemical composition analysis and determination of gas-water ratios of pressurized reservoir water. The mobile gas-water flash separator apparatus described herein is controlled by two high pressure valves; pressurized reservoir water is separated within a glass trap, and liberated gases from the sample water is captured by a cylinder fluidly connected to the manifold.

Advantageously, the mobile gas-water flash separator apparatus is capable of capturing trace volumes of gas from reservoir water for samples exhibiting low gas-water ratios (<25 standard cubic feet per barrel (SCF/bbl)) so that the liberated gas can be analyzed for chemical composition. This is accomplished by connecting a cylinder directly to the glass trap. To improve safety features, a hydrogen sulfide detector may also be connected to the apparatus. The mobile nature of the gas-water flash separator apparatus further permits ready movement about a wellsite or laboratory and ready connectability to gas chromatograph instrumentation to determine the chemical composition of liberated gas.

It is to be noted that while the present disclosure refers to the mobile separator apparatus, methods, and systems as determining gas-water ratio, the mobile separator apparatus, methods, and systems may additionally be used to determine gas-oil ratio (e.g., <25 SCF/bbl) of produced hydrocarbons, without departing from the scope of the present disclosure.

Referring now to the FIGURE, illustrated is a schematic of an example mobile gas-water flash separator system 100 (or “system 100”), according to one or more embodiments of the present disclosure. In general, the system 100 is operable at atmospheric conditions and provides a single-phase, pressurized means for flashing reservoir water to atmospheric conditions and separating flashed reservoir water into gas and water phases in a glass trap manifold. The gas volume (e.g., <25 SCF/bbl) is measured using an enclosed gasometer and liberated gas is captured in a cylinder connected directly to the glass trap. The captured liberated gas may thereafter be analyzed to determine its chemical composition.

As shown in the FIGURE, system 100 comprises a base 102. In at least one embodiment, the base 102 may be mounted to a plurality of wheels 104 (e.g., casters), thus converting the system 100 into a mobile system. While two (2) wheels 104 are shown in the FIGURE, more than two wheels 104 are contemplated for use with the mobile system 100, such as three (3) wheels 104, four (4) wheels 104, or more. In some instances, the system 100 may have between two (2) wheels and ten (10) wheels, depending on the configuration of the base and other components described herein, without departing from the scope of the present disclosure.

The base 102 may be made of any rigid material capable of providing a sturdy platform for holding at least an enclosed flash separator assembly 106 and a gasometer 108, and capable of use under wellsite conditions (e.g., environmental conditions, such as corrosion). Suitable examples of materials for forming the base 102 include a metal (e.g., carbon steel, stainless steel, and aluminum), a polymer, a thermoplastic, a composite material, or any combination thereof.

The system 100 can include a variety of flow lines (e.g., pipes, conduits, tubing, etc.), valves, and collection cylinders. In some embodiments, some or all of the flow lines, valves, and collection cylinders may be manufactured with SULFINERT® (SilcoTek® Corporation, Pennsylvania) material, which is resistant to hydrogen sulfide and carbon dioxide, and further comprises a high pressure-temperature rating.

The system 100 includes an inlet water line 109, which is in fluid communication with a flash separator line 118. It is to be noted that while the inlet water line 109 and flash separator line 118 are described as separate lines, they may be a single line, without departing from the scope of the present disclosure. Herein, the flash separator line 118 is depicted in so as to visually show water entering the glass trap 120. The flash separator line 118 extends to and fluidly communicates with a glass trap 120 of the flash separator assembly 106, and a gasometer flow line 128 extends from the glass trap 120 and to the gasometer 108. As described below, a vacuum valve 132a may be arranged in the gasometer flow line 128 and may be operable to draw gases into the gasometer flow line 128 from the glass trap 120, and may further be operable to purge (or evacuate) the entire system before and after testing of a reservoir water sample.

In at least one embodiment, a helium valve 134 may be arranged in the gasometer flow line 128, and may be used for flushing and filling the flow lines of the system 100 with helium prior to testing a of a reservoir water sample. The helium flush allows any trapped gases, such as air and/or carryover gases from previous reservoir water testing with the system 100, to be fully removed from the system 100 by opening exit valve 152 (described below). The helium fill allows avoidance of any dead volume within the system 100 (e.g., valves, lines, the glass trap, and the like) and allows gas volume to be recorded by gasometer 108 without the need to correct for any such dead volume.

The inlet water line 109 receives a pressurized reservoir fluid, such as reservoir water, from a sample cylinder 107. The reservoir fluid may have been previously collected into the sample cylinder 107 in the field from a subterranean formation, such as a water reservoir. The sample cylinder 107 may not form part of the system 100 of the present disclosure, but can be fluidly connected thereto. The sample cylinder 107 may be pressurized to monophasic conditions of the reservoir water using a positive displacement pump (not shown), and the pressurized reservoir water is received into the inlet water line 109 from a bottom portion of the sample cylinder 107. The pressurized reservoir water is received into the inlet water line 109 from the bottom portion of the sample cylinder 107 because the sample cylinder 107 contains a piston separating a back-pressure portion from a sample portion (i.e., there is no fluid communication between the back-pressure portion and the sample portion due to the piston). The back-pressure portion is filled with a pressurizing fluid, such as glycol, to pressurize the reservoir water to monophasic conditions and the sample portion is required to be positioned downward to allow for gravity separation of the pressurized reservoir water (i.e., the pressurized reservoir water will settle to the bottom of the sample cylinder due to its relative higher density).

The pressurized reservoir water is received into the inlet water line 109 for delivery to the flash separator assembly 106 through two in-line high-pressure valves 110 and 112. Purge valve 150, described below, in combination with in-line high-pressure valves 110 and 112, form a three-way, two-stem connection valve system that permits purging of pressurized reservoir water by closing in-line pressure valve 110 and opening purge valve 150 without the pressurized reservoir water entering through in-line high-pressure valve 112. In-line high-pressure valve 112 is used to control the rate (speed) and pressure of the flow of the pressurized reservoir water. Moreover, if the in-line high-pressure valve 112 fails, in-line high-pressure valve 110 can be closed. The pressurized reservoir water may be introduced into the flash separator assembly 106 using the valves 110, 112, which ensure that the pressure of the incoming reservoir water does not overload (over pressurize) the flash separator assembly 106. In one or more aspects, each valve 110, 112 may be rated to 30,000 pounds per square inch (psi), although valves with other pressure ratings may be used system 100, without departing from the scope of the disclosure. A pressure gauge 114, such as a 30,000 psi pressure gauge, may be located between the first and second valves 110, 112 and may be operable to monitor pressure of the reservoir fluid (water) as it traverses the valves 110, 112 via inlet water line 109.

The incoming reservoir water enters the flash separator assembly 106 via the inlet water line 109, which may feed into the flash separator line 118. The inlet water line 109 and the flash separator line 118 may be made of the same or different material, and may exhibit the same or different diameter. In at least one embodiment, the inlet water line 109 and the flash separator line 118 may comprise the same flow line, and the flash separator line 118 may merely provide an extension of the inlet water line 109. As illustrated, the flash separator assembly 106 includes the glass trap 120 with a manifold 116 secured thereto. The flash separator line 118 may penetrate the manifold 116 to discharge the pressurized reservoir water into the glass trap 120 at atmospheric conditions, shown in the FIGURE as reservoir water 124.

The glass trap 120 may exhibit a variety of suitable sizes and volumes, depending on operational parameters. In some aspects, the volume of the glass trap 120 may range between about 250 milliliters (mL) and about 1000 mL, such as a 250 mL glass trap or a 500 mL glass trap. The glass trap 120 is preferably composed of glass because reservoir waters comprise, among other components, salts and hydrogen sulfide that are reactive with other materials (e.g., plastics). Moreover, glass is easily cleanable with organic solvents, for example, for use in subsequent operations.

In operation, the pressurized reservoir water 124 received in the flash separator assembly 106 is separated within the glass trap 120 and stabilized to atmospheric conditions, thereby allowing flashed gas 122 to be liberated from the deposited reservoir water 124, which remains at the bottom of the glass trap 120. The flashed gas 122 may be composed of various compounds such as, but not limited to, non-hydrocarbon compounds (e.g., nitrogen, carbon dioxide, hydrogen sulfide, and the like) and hydrocarbon compounds (e.g., methane, ethane, propane, iso-butane, normal-butane, iso-pentane, normal-pentane, hexanes, heptanes, octanes, nonanes, decanes, and the like) (see Table 2 below).

In one or more embodiments, the system 100 further includes a first flashed gas cylinder 126 in fluid communication with the glass trap 120 through the manifold 116. More specifically, the first flashed gas cylinder 126 may be arranged in the gasometer flow line 128 and may receive the flashed gas 122 from the glass trap 120. In some embodiments, the first flashed gas cylinder 126 may comprise a small volume cylinder (e.g., about 50 mL to about 75 mL) that captures and collects the flashed gas 122, but could alternatively be a variety of other sizes. As shown, a lower valve 126a and an upper valve 126a may be arranged at opposing fluid ends of the first flashed gas cylinder 126. To collect the flashed gas 122 within the first flashed gas cylinder 126 and simultaneously permit flow to the gasometer 108, both of the valves 126a,b are opened.

After measurements are made by the gasometer 108, both valves 126a,b on the first flashed gas cylinder 126 may be closed, thus trapping a portion of the flashed gas 122 within the first flashed gas cylinder 126. The first flashed gas cylinder 126 may then be removed from the system 100 and fluidly coupled to a gas chromatograph instrument 146 to undertake a gas chromatography assessment of the flashed gas 122 and thereby determine the composition of the flashed gas 112. At least one advantage of fluid communication (e.g., by connecting or mechanical means, such as bolding) between the first flashed gas cylinder 126 and the glass trap 120 through the manifold 116 is to capture small or trace volumes of the flashed gas 122, which is chemically evaluated for composition required to quantify the presence of hydrocarbons and/or non-hydrocarbons.

The gasometer flow line 128 may include a variety of mechanical elements arranged therein to undertake a variety of operations. In some embodiments, for example, a second or “duplicate” flashed gas cylinder 130 may be in fluid communication with the gasometer flow line 128. The duplicate flashed gas cylinder 130 may be a small volume cylinder (e.g., about 50 mL to about 75 mL) configured to capture and collect a portion of the flashed gas 122 circulating within the gasometer flow line 128. As shown, the duplicate flashed gas cylinder 130 includes a lower valve 130a and an upper valve 130a. To permit flow to the gasometer 108, while simultaneously collecting flashed gas 122 within the duplicate flashed gas cylinder 130, the lower valve 130a is opened and upper valve 130b is closed. After measurements are made by the gasometer 108, the lower valve 130a is closed, thus capturing a portion of the flashed gas 122 within the duplicate flashed gas cylinder 130 to be analyzed for gas chromatography assessment to determine the composition of the flashed gas 112.

In some embodiments, the vacuum valve 132a and the vacuum pressure gauge 132b may be arranged downstream from the duplicate flashed gas cylinder 130 within the gasometer flow line 128. The vacuum valve 132a may be operable to generate a vacuum in the gasometer flow line 128, which draws the flashed gas 122 out of the glass trap 120 and into the gasometer flow line 128. The vacuum generated by the vacuum valve 132a may be in the range of −14.7 psi to +50 psi, and encompassing any value and subset therebetween. Note that negative pressure is maintained for the system 100 before helium flushing and filling and the positive pressure is used to monitor the helium flush and fill pressure. In at least one embodiment, prior to testing a pressurized reservoir water with the system 100, the vacuum valve 132a can be opened to pull a vacuum through the gasometer 108, the gasometer flow line 128, the glass trap 120, and the inlet water and flash separator lines 109, 118. The vacuum pressure gauge 132b may be used to monitor the pressure within the gasometer flow line 128. The vacuum valve 132a may further be operable to aid in flushing and filling the various flow lines of the system 100 with helium using helium valve 134.

The flashed gas 122 flowing through the gasometer flow line 128 may be conveyed to the gasometer 108 via a valve 136. The gasometer 108 is used to determine the volume, temperature, and pressure of the flashed gas 122 in order to calculate gas-water ratio. The gasometer 108 is enclosed and comprises a gas volume meter 138 designed to determine the volume of the flashed gas 122. The gas volume meter 138 may comprise, for example, a 1000 mL volume meter.

The gasometer 108 may further include a gas temperature gauge 140 configured to determine the real-time temperature of the flashed gas 122, and a gas pressure gauge 142 may be included to determine the real-time pressure of the flashed gas 122 received by the gasometer 108.

The gas-water ratio of the reservoir fluid introduced into the system 100 may be determined by weighing the reservoir water 124 within the glass trap 120 after testing with the system 100 is complete as compared to the initial weight of the glass trap 120 (clean, having no contents therein and prior to conducting any testing with the system 100 (i.e., before vacuum and helium flush and fill)) and the volume of the gas recorded by the gas volume meter 138 of the gasometer 108. The gas-water ratio is the ratio of volume of flashed gas 122 to the volume of reservoir water 124. Other measurements include the specific gravity of the flashed gas 122 and the density of the reservoir water 124 collected within the glass trap 120. The gasometer 108 further includes an exit valve 152 operable to purge the flashed gas 122 from the gasometer 108 and/or the flow lines of the system 100 as a whole.

The system 100 may optionally include a hydrogen sulfide detector 144 operatively coupled to the enclosed flash separator assembly 106. While the hydrogen sulfide detector 144 is shown at an upper (top) location of the perimeter of the enclosed flash separator assembly 106, it may be located at any location on or within the flash separator assembly 106 (e.g., upper, right side, or left side, and at any horizontal, vertical, or width), without departing from the scope of the present disclosure. The hydrogen sulfide detector 144 operates as a safety device to detect any leaks of the toxic acid gas within the enclosed flash separator assembly 106, and thus the system 100 as a whole. The hydrogen sulfide detector 144 can measure hydrogen sulfide in an amount of from 0 parts per million (ppm) to 25 ppm, and encompassing any value and subset therebetween.

In some embodiments, the system 100 may further include a purge line 148 and associated purge valve 150. The purge line 148 may be fluidly coupled to the inlet water line 109 (or the flash separator line 118) and may be configured as an outlet to extract a sample of the reservoir water from 107, such as to remove any sediment and/or solids therefrom. The purge valve 150 may be operable to allow the pressurized reservoir water 107 to be purged through purge line 148 by closing in-line high-pressure valve 110 and preventing any said sediment and/or solids from entering in-line high-pressure valve 112.

After the volume, temperature, and pressure measurements of the flashed gas 122 are recorded, valves 126a, 126b on the first flashed gas cylinder 126 may be closed and valve 130a of the duplicate flashed gas cylinder 130 may be closed. One or both of the flashed gas cylinders 126, 130 may then be removed or otherwise placed in fluid communication with one or more gas chromatograph instruments 146 (one shown), The collected flashed gas 122 present in each cylinder 126, 130 may be tested using the gas chromatograph instrument 146 to determine the contents of the collected flashed gas 122.

Accordingly, the present disclosure provides a system comprising the system 100 and the gas chromatograph instrument 146. The present disclosure thus provides a method of determining both the gas-water ratio of a flashed gas using the mobile gas-water flash separator system 100, and the composition of the same flashed gas using a gas chromatograph instrument 146.

Methods of the present disclosure include utilizing the mobile flashing pressurized reservoir water from monophasic conditions to atmospheric conditions and separating the atmospheric monophasic reservoir water into a water phase and a gas phase (liberated, flashed gas). The volume, temperature, and pressure of the flashed gas are measured using a digital gasometer. The specific gravity of the flashed gas may be measured. The weight of the water phase, as well as its density, are also measured. The obtained measurements are used to determine atmospheric gas-water ratio. The flashed gas composition is further analyzed to C₉₊ using a gas chromatograph instrument.

Referring again to the FIGURE, methods described herein include pressurizing collected reservoir water in sample cylinder 107 to monophasic conditions using a positive displacement pump (not shown). As noted above, the sample portion of the sample cylinder 107 is facing downwards. The sample cylinder 107 is connected for fluid communication with in-line high-pressure valves 110, 112 and purge valve 150 (the three-way, two-stem connection valve system described above) using inlet water line 109. The clean, empty glass trap 120 is detached (or is already detached) from the system 100 and weighed (the “initial” glass trap 120 weight), and thereafter attached to the manifold 116 as an integral part of the system 100. Valves 110, 112, 126a, 126b, 130a, 132a, 132b, and 136 are opened and valves 150, 130b, 134, and 152 are closed. The system 100 is evacuated using vacuum valve 132a until vacuum pressure gauge 132b reads—14.7 psi. Valves 110, 112, and 132a are closed and the system is slowly flushed with helium by opening valves 134 and 152. Thereafter, valve 152 is closed and the system is filled with helium, followed by closing valve 134. An initial gas volume is recorded using the gas volume meter 138 of the gasometer 108.

Pressurized reservoir water from the sample cylinder 107 is flowed into the inlet water line 109 to closed valve 110. At least a portion of the pressurized reservoir water may be purged through purge line 148 by opening purge valve 150 to remove any sediment and/or solids therefrom. It is to be noted that valve 110 is to remain closed during any purging. After purging, if applicable, the purge valve 150 is closed. The pressurized reservoir water is loaded further into the system 100 by opening valve 110; the pressure of the pressurized reservoir water is monitored using pressure gauge 114, which should be identical or near identical to the pressure of the pressurized reservoir water in the sample chamber 107. Valve 112 is slowly opened to maintain the pressurized reservoir water at monophasic conditions and allowed to flow through flash separator line 118 (which may be identical to inlet water line 109) and into the glass trap 120 to separate into flashed gas 122 and reservoir water 124 at atmospheric conditions. Once the glass trap 120 is full of reservoir water 124, the separation process is stopped by closing valve 112. Thereafter valves 110, 126a. 126b. and 130a are closed. The final gas volume is measured using the gas volume meter 138 of the gasometer 108, as well as its pressure and temperature using gasometer 108. The glass trap 120 is removed and weight (the “final” weight of the glass trap 120). At least the first flashed gas cylinder 126 is removed and the chemical composition of the flashed gas 122 determined using the gas chromatograph instrument 146. If flashed gas 122 is also collected in the second flashed gas cylinder 130, it may additionally be removed and the chemical composition of the flashed gas 122 therein determined using the gas chromatograph instrument 146. The density of the water phase 124 may further be determined.

The results of the gasometer measurements and water weight measurements may be presented in a data sheet. Such measurements may include the flashed gas phase data from the gasometer results—an initial pressure (Pi) in psi of the gasometer upon detection of flashed gas; a final pressure (Pf) in psi of the gasometer after final collection of the flashed gas; an initial volume (Vi) in mL of the gasometer upon detection of flashed gas; an final volume (Vf) of the gasometer after final collection of the flashed gas; and a temperature in ° ° C. of the gasometer after final collection of flashed gas. The water phase data from weighing the glass trap before and after analysis of the pressurized water sample may also be included, comprising an initial weight in grams (g) of the glass trap and a final weight in g of the glass trap containing the collected water. The density of the water may also be measured in grams per mL and reported on the data sheet. Further, details such wellbore identity, wellbore depth, formation identity, formation type, formation pressure in psi, formation temperature in ° C. or ° F., sample identity (e.g., sample number), sample cylinder identity (e.g., sample cylinder number), sample date, and analysis date may be included. Finally, the data sheet may include the signature of the person that performed the analysis.

### Example Embodiments

To facilitate a better understanding of the embodiments described herein, the following examples of various representative embodiments are given. In no way should the following examples be read to limit, or to define, the scope of the present disclosure.

### Example

In this Example, a representative sample of reservoir water was tested using the mobile gas-water ratio sampler apparatus of the present disclosure, followed by gas chromatography to determine the composition of the liberated flashed gas. The mobile gas-water ratio sampler apparatus results are shown in Table 1, including measurements from the gasometer represented as the “gas phase data” and measurements from the initial and final weight of the glass trap represented as “water phase data.” The gas phase data further includes the specific gravity of the flashed gas, and the water phase data further includes the density of the obtained water phase from the glass trap. Finally, the gas-water ratio is calculated. The results of the gas chromatograph testing of the gas phase (liberated flashed gas) is shown in Table 2.

TABLE 1
GAS PHASE DATA

| | |
|---|---|
| Initial Pressure (psi) | 14.9 |
| Final Pressure (psi) | 14.9 |
| Initial Volume (mL) | 0.93 |
| Final Volume (mL) | 400 |
| Temperature (° C.) | 25.8 |
| Specific Gravity (Air = 1) | 0.7123 |

WATER PHASE DATA

| | |
|---|---|
| Initial Weight of Glass trap (g) | 225.55 |
| Final Weight of Glass trap (g) | 550.35 |
| Density @ 60° F. (g/mL) | 1.1245 |

GAS-WATER RATIO (SCF/BBL)
2

TABLE 2
GAS PHASE DATA

| Component | Mole % |
|---|---|
| Nitrogen | 0.10 |
| Carbon Dioxide | 6.00 |
| Hydrogen Sulfide | 3.00 |
| Methane | 90.44 |
| Ethane | 0.30 |
| Propane | 0.10 |
| i-Butane | 0.05 |
| n-Butane | 0.01 |
| i-Pentane | 0.00 |
| Hexanes | 0.00 |
| Heptanes | 0.00 |
| Octanes | 0.00 |
| Nonanes | 0.00 |
| Decanes | 0.00 |
| Total | 100.00 |

Accordingly, the mobile gas-water separator apparatus of the present disclosure provides for gas-water ratio testing at or near a wellsite that is sensitive to detection of low gaseous concentrations in formation water samples and is easily connectable to gas chromatograph instrumentation.

Embodiments disclosed herein include:

Embodiment A: A gas-water flash separator system, comprising: an inlet water line fluidly communicable with a sample cylinder containing a pressurized reservoir fluid; a glass trap sealable with a manifold and in fluid communication with the inlet water line to receive the pressurized reservoir fluid, the pressurized reservoir fluid being separable within the glass trap at atmospheric conditions into a flashed gas and a reservoir water; and a gasometer in fluid communication with the glass trap via a gasometer flow line and operable to receive the flashed gas and measure a volume, a temperature, and a pressure of the flashed gas, wherein a weight and a density of the reservoir water is measured and compared against the volume of the flashed gas to thereby determine a gas-water ratio of the pressurized reservoir fluid.

Embodiment B: A system comprising: a base including a plurality of wheels; an enclosed flash separator assembly positioned on the base and including: an inlet water line fluidly communicable with a sample cylinder containing a pressurized reservoir fluid; a glass trap sealable with a manifold and in fluid communication with the inlet water line to receive the pressurized reservoir fluid, the pressurized reservoir fluid being separable within the glass trap at atmospheric conditions into a flashed gas and a reservoir water; and a gasometer positioned on the base and in fluid communication with the glass trap via a gasometer flow line, the gasometer being operable to receive the flashed gas and measure a volume, a temperature, and a pressure of the flashed gas, wherein a weight and a density of the reservoir water is measured and compared against the volume of the flashed gas to thereby determine a gas-water ratio of the pressurized reservoir fluid.

Embodiment C: A method comprising: fluidly coupling a sample cylinder containing a pressurized reservoir fluid to an inlet water line; receiving the pressurized reservoir fluid in the inlet water line, and conveying the pressurized reservoir fluid to a glass trap sealed with a manifold; separating the pressurized reservoir fluid in the glass trap at atmospheric conditions into a flashed gas and a reservoir water; receiving the flashed gas in a gasometer flow line in fluid communication with the glass trap via a gasometer flow line; measuring a volume, a temperature, and a pressure of the flashed gas with the gasometer; measuring a weight and a density of the reservoir water; and comparing the weight and the density against the volume of the flashed gas and thereby determining a gas-water ratio of the pressurized reservoir fluid.

Embodiments A through C may include one or more of the following additional elements, as described hereinbelow.

Element 1: wherein the glass trap and the gasometer are arranged on a base and the base includes a plurality of wheels that provides mobility to the system.

Element 2: further comprising: a flashed gas cylinder arranged in the gasometer flow line; a first valve arranged at a first fluid end of the flashed gas cylinder; a second valve arranged at a second fluid end of the flashed gas cylinder, wherein closing the first and second valves captures a sample of the flashed gas within the flashed gas cylinder; and a gas chromatograph instrument fluidly communicable with the flashed gas cylinder to determine a composition of the sample of the flashed gas.

Element 3: further comprising: a flashed gas cylinder arranged in the gasometer flow line; a first valve arranged at a first fluid end of the flashed gas cylinder; a second valve arranged at a second fluid end of the flashed gas cylinder, wherein closing the first and second valves captures a sample of the flashed gas within the flashed gas cylinder; and a gas chromatograph instrument fluidly communicable with the flashed gas cylinder to determine a composition of the sample of the flashed gas, wherein the flashed gas cylinder is removable from the gasometer flow line to place the flashed gas cylinder in fluid communication with the gas chromatograph instrument.

Element 4: further comprising: a flashed gas cylinder arranged in the gasometer flow line; a first valve arranged at a first fluid end of the flashed gas cylinder; a second valve arranged at a second fluid end of the flashed gas cylinder, wherein closing the first and second valves captures a sample of the flashed gas within the flashed gas cylinder; and a gas chromatograph instrument fluidly communicable with the flashed gas cylinder to determine a composition of the sample of the flashed gas, wherein the flashed gas cylinder comprises a first flashed gas cylinder and the sample of the flashed gas comprises a first sample of the flashed gas, the system further comprising: a second flashed gas cylinder in fluid communication with the gasometer flow line downstream from the first flashed gas cylinder; a third valve interposing the gasometer flow line and the second flashed gas cylinder, wherein closing the third valve captures a second sample of the flashed gas within the second flashed gas cylinder, and wherein the gas chromatograph instrument is fluidly communicable with the second flashed gas cylinder to determine a composition of the second sample of the flashed gas.

Element 5: further comprising at least one high-pressure valve arranged in the inlet water line and interposing the sample cylinder and the glass trap, the at least one high-pressure valve being operable to regulate a pressure of the pressurized reservoir fluid conveyed to the glass trap.

Element 6: further comprising a vacuum valve arranged in the gasometer flow line and operable to create a vacuum in the gasometer flow line that draws the flashed gas from the glass trap and into the gasometer flow line.

Element 7: further comprising a vacuum pressure gauge for monitoring a pressure within the gasometer flow line.

Element 8: further comprising a helium valve in fluid communication with the gasometer flow line and operable to inject helium into the gasometer flow line.

Element 9: wherein the enclosed flash separator assembly further includes a hydrogen sulfide detector.

Element 10: further comprising: receiving a sample of the flashed gas within a flashed gas cylinder arranged in the gasometer flow line; closing first and second valves arranged at opposing first and second fluid ends, receptively, of the flashed gas cylinder and thereby capturing the sample of the flashed gas within the flashed gas cylinder; placing the flashed gas cylinder in fluid communication with a gas chromatograph instrument; and determining a composition of the sample of the flashed gas with the gas chromatograph instrument.

Element 11: further comprising: receiving a sample of the flashed gas within a flashed gas cylinder arranged in the gasometer flow line; closing first and second valves arranged at opposing first and second fluid ends, receptively, of the flashed gas cylinder and thereby capturing the sample of the flashed gas within the flashed gas cylinder; placing the flashed gas cylinder in fluid communication with a gas chromatograph instrument; and determining a composition of the sample of the flashed gas with the gas chromatograph instrument, wherein determining the composition of the sample of the flashed gas comprises measuring for at least one of Nitrogen, Carbon Dioxide, Hydrogen Sulfide, Methane, Ethane, Propane, i-Butane, n-Butane, i-Pentane, Hexanes, Heptanes, Octanes, Nonanes, Decanes.

Element 12: wherein receiving the pressurized reservoir fluid in the inlet water line comprises regulating a pressure of the pressurized reservoir fluid conveyed to the glass trap using at least one high-pressure valve arranged in the inlet water line and interposing the sample cylinder and the glass trap.

Element 13: wherein at least the glass trap and the gasometer are mounted to a base including a plurality of wheels.

Embodiment A may have one or more of additional Elements 1-8 in any one, more, or all non-limiting combinations.

Embodiment B may have one or more of additional Elements 1-9 in any one, more, or all non-limiting combinations.

Embodiment C may have one or more of additional Elements 10-13 in any one, more, or all non-limiting combinations.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, applications, elements, and/or components, but do not preclude the presence or addition of one or more other features, integers, steps, applications, elements, components, and/or groups thereof.

Terms of orientation used herein are merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and is not limited to either unless expressly referenced as such.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

The invention claimed is:

1. A system, comprising:
an inlet water line fluidly communicable with a sample cylinder containing a pressurized reservoir fluid;
a glass trap sealable with a manifold and in fluid communication with the inlet water line to receive the pressurized reservoir fluid, the pressurized reservoir fluid being separable within the glass trap at atmospheric conditions into a flashed gas and a reservoir water; and
a gasometer in fluid communication with the glass trap via a gasometer flow line and operable to receive the flashed gas and measure a volume, a temperature, and a pressure of the flashed gas,
wherein a weight and a density of the reservoir water is measured and compared against the volume of the flashed gas to thereby determine a gas-water ratio of the pressurized reservoir fluid.

2. The system of claim 1, wherein the glass trap and the gasometer are arranged on a base and the base includes a plurality of wheels that provides mobility to the system.

3. The system of claim 1, further comprising:
a flashed gas cylinder arranged in the gasometer flow line;
a first valve arranged at a first fluid end of the flashed gas cylinder;
a second valve arranged at a second fluid end of the flashed gas cylinder, wherein closing the first and second valves captures a sample of the flashed gas within the flashed gas cylinder; and
a gas chromatograph instrument fluidly communicable with the flashed gas cylinder to determine a composition of the sample of the flashed gas.

4. The system of claim 3, wherein the flashed gas cylinder is removable from the gasometer flow line to place the flashed gas cylinder in fluid communication with the gas chromatograph instrument.

5. The system of claim 3, wherein the flashed gas cylinder comprises a first flashed gas cylinder and the sample of the flashed gas comprises a first sample of the flashed gas, the system further comprising:
a second flashed gas cylinder in fluid communication with the gasometer flow line downstream from the first flashed gas cylinder;
a third valve interposing the gasometer flow line and the second flashed gas cylinder, wherein closing the third valve captures a second sample of the flashed gas within the second flashed gas cylinder, and
wherein the gas chromatograph instrument is fluidly communicable with the second flashed gas cylinder to determine a composition of the second sample of the flashed gas.

6. The system of claim 1, further comprising at least one high-pressure valve arranged in the inlet water line and interposing the sample cylinder and the glass trap, the at least one high-pressure valve being operable to regulate a pressure of the pressurized reservoir fluid conveyed to the glass trap.

7. The system of claim 1, further comprising a vacuum valve arranged in the gasometer flow line and operable to create a vacuum in the gasometer flow line that draws the flashed gas from the glass trap and into the gasometer flow line.

8. The system of claim 7, further comprising a vacuum pressure gauge for monitoring a pressure within the gasometer flow line.

9. The system of claim 1, further comprising a helium valve in fluid communication with the gasometer flow line and operable to inject helium into the gasometer flow line.

10. A system comprising:
a base including a plurality of wheels;
an enclosed flash separator assembly positioned on the base and including:
an inlet water line fluidly communicable with a sample cylinder containing a pressurized reservoir fluid;
a glass trap sealable with a manifold and in fluid communication with the inlet water line to receive the pressurized reservoir fluid, the pressurized reservoir fluid being separable within the glass trap at atmospheric conditions into a flashed gas and a reservoir water; and
a gasometer positioned on the base and in fluid communication with the glass trap via a gasometer flow line, the gasometer being operable to receive the flashed gas and measure a volume, a temperature, and a pressure of the flashed gas,
wherein a weight and a density of the reservoir water is measured and compared against the volume of the flashed gas to thereby determine a gas-water ratio of the pressurized reservoir fluid.

11. The system of claim 10, further comprising:
a flashed gas cylinder arranged in the gasometer flow line;
a first valve arranged at a first fluid end of the flashed gas cylinder;
a second valve arranged at a second fluid end of the flashed gas cylinder, wherein closing the first and second valves captures a sample of the flashed gas within the flashed gas cylinder; and
a gas chromatograph instrument fluidly communicable with the flashed gas cylinder to determine a composition of the sample of the flashed gas.

12. The system of claim 11, wherein the flashed gas cylinder comprises a first flashed gas cylinder and the sample of the flashed gas comprises a first sample of the flashed gas, the system further comprising:
a second flashed gas cylinder in fluid communication with the gasometer flow line downstream from the first flashed gas cylinder;
a third valve interposing the gasometer flow line and the second flashed gas cylinder, wherein closing the third valve captures a second sample of the flashed gas within the second flashed gas cylinder, and
wherein the gas chromatograph instrument is fluidly communicable with the second flashed gas cylinder to determine a composition of the second sample of the flashed gas.

13. The system of claim 10, wherein the enclosed flash separator assembly further includes a hydrogen sulfide detector.

14. The system of claim 10, further comprising a vacuum valve arranged in the gasometer flow line and operable to create a vacuum in the gasometer flow line that draws the flashed gas from the glass trap and into the gasometer flow line.

15. The system of claim 10, further comprising at least one high-pressure valve arranged in the inlet water line and interposing the sample cylinder and the enclosed flash separator assembly, the at least one high-pressure valve being operable to regulate a pressure of the pressurized reservoir fluid conveyed to the glass trap.

16. A method comprising:
fluidly coupling a sample cylinder containing a pressurized reservoir fluid to an inlet water line;
receiving the pressurized reservoir fluid in the inlet water line, and conveying the pressurized reservoir fluid to a glass trap sealed with a manifold;
separating the pressurized reservoir fluid in the glass trap at atmospheric conditions into a flashed gas and a reservoir water;
receiving the flashed gas in a gasometer flow line in fluid communication with the glass trap via a gasometer flow line;
measuring a volume, a temperature, and a pressure of the flashed gas with the gasometer;
measuring a weight and a density of the reservoir water; and
comparing the weight and the density against the volume of the flashed gas and thereby determining a gas-water ratio of the pressurized reservoir fluid.

17. The method of claim 16, further comprising:
receiving a sample of the flashed gas within a flashed gas cylinder arranged in the gasometer flow line;
closing first and second valves arranged at opposing first and second fluid ends, receptively, of the flashed gas cylinder and thereby capturing the sample of the flashed gas within the flashed gas cylinder;
placing the flashed gas cylinder in fluid communication with a gas chromatograph instrument; and
determining a composition of the sample of the flashed gas with the gas chromatograph instrument.

18. The method of claim 17, wherein determining the composition of the sample of the flashed gas comprises measuring for at least one of Nitrogen, Carbon Dioxide, Hydrogen Sulfide, Methane, Ethane, Propane, i-Butane, n-Butane, i-Pentane, Hexanes, Heptanes, Octanes, Nonanes, Decanes.

19. The method of claim 16, wherein receiving the pressurized reservoir fluid in the inlet water line comprises regulating a pressure of the pressurized reservoir fluid conveyed to the glass trap using at least one high-pressure valve arranged in the inlet water line and interposing the sample cylinder and the glass trap.

20. The method of claim 16, wherein at least the glass trap and the gasometer are mounted to a base including a plurality of wheels.
